(19) United States
(12) Patent Application Publication
(10) Pub. No.: US 2004/0170687 A1
(43) Pub. Date: Sep. 2, 2004

(54) Compositions with improved stability and methods of formulation thereof

(75) Inventors: William R. Hurd, Noblesville, IN (US); Michael J. Dempsey, Columbus, IN (US); William C. Williams, Indianapolis, IN (US)

Correspondence Address: WOOD, HERRON & EVANS, LLP, 2700 CAREW TOWER, 441 VINE STREET, CINCINNATI, OH, 45202, US

(73) Assignee: Integrity Pharmaceutical Corporation, Fishers, IN

(21) Appl. No.: 10/375,391

(22) Filed: Feb. 27, 2003

(51) Int. Cl. A61K 9/20; A61K 9/24

(52) U.S. Cl. 424/471

(57) ABSTRACT

Ingredients in pharmaceutical compositions are provided with a coating material for improving stability thereof. The coating material prevents reactive contact between otherwise incompatible ingredients in the composition. Without contact, incompatible ingredients will not degrade thereby prolonging the stability and shelf life of the composition.

### BACKGROUND OF THE INVENTION

I. Field of the Invention

The present invention relates to improving the stability of a composition, and in particular, to prolonging the shelf life of a composition having two or more incompatible ingredients.

II. Description of the Prior art

There are literally tens of thousands of prescription and non-prescription, or “over-the-counter”, medications or pharmaceutical compositions available for use. Ingestible compositions in suitable pharmaceutically accepted formulations, such as a liquid, a pill, a tablet, a caplet, or a capsule including soft, hard and gelatin capsules, and non-ingestible compositions, such as a dermal patch, generally include a multitude of ingredients, one or more of which are typically “active” ingredients. A pharmaceutical active ingredient is a substance having a specific physiological action and effect with minimal or no side effects. Thus, active ingredients are generally targeted for the treatment of specific symptoms, ailments, diseases, or disorders related to the body. Non-active ingredients, such as lubricants, colorants, flavoring agents, texture modifiers, disintegrants, preservatives, among others, generally referred to as “excipients”, are commonly included with the active ingredient(s) to render the composition desirable in terms of its physical properties and aesthetic characteristics.

It is often desirable to combine multiple ingredients in a single pharmaceutically acceptable composition. Inclusion of multiple ingredients in a single composition generally reduces costs and provides the convenience of consuming a single medication rather than multiple medications for treating individual symptoms. This is especially true with compositions including a combination of active ingredients. For example, a single medication including multiple active ingredients may be administered for treating allergy symptoms, such as a runny nose, a fever, a cough, and inflammation, wherein each active ingredient is directed towards a specific allergy symptom. Further, the medication may include an active to promote drowsiness and rest necessary for recovery from the allergy thereby relieving the patient of the effort to ingesting a separate medication for sleep. However, a combination of ingredients, either actives or non-actives, is not without drawbacks. For example, the stability of a composition may be compromised due to the inclusion of a combination of two or more incompatible ingredients.

Incompatibility between ingredients is but one of many factors affecting the stability and, therefore, the shelf life of a composition. Incompatibility generally depends upon the level and type of interaction between individual ingredients in the composition. The interaction may be physical, wherein one ingredient is harmfully reactive with another ingredient resulting from physical contact between the two ingredients in the composition. Particularly, where physical contact over a prolonged period of time leads to a mild or slow reaction between ingredients, the effects of the interaction may be harmful and cause gradual degradation of one or both ingredients in the composition. Generally, the degradation is chemical, i.e., one or both of the ingredients are altered at a chemical level thereby affecting its activity as well as the overall stability and usefulness of the composition. Chemical interactions between ingredients without physical contact may also cause degradation of one or both ingredients.

Stability issues resulting from incompatibility generally adversely affect the costs of commercializing the composition. Before a formulated composition or medication may be made commercially available for public consumption and use in the United States it must pass strict stability standards set forth by the United States Food and Drug Administration (FDA) and United States Pharmacopeia (USP). Particularly, the FDA requires that a medication have a certain level of stability over a period of time, i.e., that ingredients in the composition collectively remain stable thereby maintaining the overall activity, function and, therefore, safety of the composition for that specified period of time. To this end, the shelf life of a particular pharmaceutical composition must also comply with both the USP and FDA requirements. Thus, a composition that gradually destabilizes over time, due to the interaction of incompatible ingredients, requires longer stability tests in compliance with government stability standards. Such tests add to the costs of the finally formulated medical composition. It would be even more costly, and undesirable, for companies who have already invested large amounts of money to design, discover, synthesize, and formulate active ingredients into a single pharmaceutically acceptable formulation, to fail such USP and FDA stability requirements. It is therefore desirable to improve the stability of pharmaceutical compositions to comply with government stability requirements.

Stability related degradation of one or more incompatible ingredients in a composition can be costly to a company, even beyond the dollars spent in developing and marketing the final formulation. Particularly, where one or more of the incompatible ingredients are active ingredients, the consequences of consuming a composition after it has remained in a “degrading” state for a period of time could be harmful. For example, degradation, caused by the deactivation or reduction of active form of one or both incompatible ingredients in the composition, may lead to the inability of the composition to effectively treat the symptom or disorder. Moreover, dangerous and potentially toxic side products resulting from the degrading ingredient(s) may be present in the composition. Accordingly, incompatibility may pose dire and possibly fatal consequences in addition to affecting the ability of the composition to successfully treat the disorder. It is, therefore, desirable to address incompatibility between ingredients, and render them less reactive, or “more compatible” in a composition.

In addition to incompatibility, the stability of a composition may also be adversely affected by environmental conditions, such as the climate in which the final composition or formulation is stored. For example, humidity, pressures, and/or temperatures are a few of the conditions that must be considered when storing these compounds. Many ingredients and/or compositions are affected from exposure to such conditions and often degrade affecting the overall stability and/or activity of the composition. Packaging is generally selected such that humidity and heat are not of much consequence to compositional stability.

The prior art has proposed methods for improving the stability of compositions including a multitude of ingredients. For example, U.S. Pat. No. 6,001,391 to Zeidler et al., discloses a process for producing solid drug forms having at least two phases comprising molding a melt of a polymeric binder with or without at least one active ingredient, and incorporating at least one solid product into the plastic composition during the molding step.

U.S. Pat. No. 6,406,745, issued to Talton, discloses a method of coating core materials with ablated particulate target materials at pressures of about 10 Torr or higher. The ablated particulate target materials were produced from ablating or vaporizing target materials for coating the core materials.

U.S. Pat. No. 5,562,642, issued to Smith et al., discloses a system for topical delivery of a plurality, preferably two, incompatible dermatological agents to the skin from a single dispensing and applicator system comprising a plurality of compartmentalized applicator pads. Thus, the incompatible agents are compartmentalized and topically delivered through the applicator system.

However, while these prior art methods separate ingredients in a composition, such compartmentalization, high temperature and high pressure ablating techniques, and independent and distinct phase separation is not only inefficient and undesirable by adding costs to the manufacturing process and the consumer, but also warrants satisfying additional government requirements prior to commercial use. Further, the high temperatures and pressures of the ablating technique may be destructive to the ingredient and/or the composition and, therefore, may not be suitable or applicable to many pharmaceutical active ingredients.

Thus, there is a need to improve the stability of compositions having two or more otherwise incompatible ingredients in a single formulation. There is also a need to do so in a manner that is mild and non-destabilizing for the ingredient. Further, there is a need to improve the overall stability and shelf life of the composition sufficient to meet or exceed government standards. Still further, there is a need to provide these compositions having improved stability in a convenient and cost effective manner.

### SUMMARY OF THE INVENTION

The present invention provides compositions with improved stability and methods of improving stability while addressing the drawbacks and weaknesses of the prior art. To this end, and in accordance with the principles of the present invention, pharmaceutical compositions having at least two ingredients, a first ingredient of which is reactive or incompatible with a second ingredient, are provided with a barrier, such as a coating, designed to reduce reactivity between the ingredients, and particularly between the first and second ingredients, in a final, pharmaceutically accepted formulation. By preventing reducing reactivity between the first and second ingredients in the formulation, the barrier minimizes degradation of one or both of the ingredients thereby improving the stability and prolonging the shelf life of the formulation.

The first and second ingredients, independently, may be any ingredient desired or needed in the formulation. For example, the first and/or second ingredient may, individually, be an active or a non-active ingredient. The present invention is useful for compositions where the first and second ingredients are “reactive” with one another or otherwise incompatible with one another in the formulation. The term “reactive”, as used herein with reference to ingredients, is intended to generally refer to incompatibility between the ingredients. For example, a first ingredient (A) which, in the physical presence of a second ingredient (B), reacts with the second ingredient (B) so as to degrade one or both of ingredients (A) and (B) over time reflects incompatibility between (A) and (B). The destabilizing affects of incompatibility can be particularly harmful where both ingredients are biologically active ingredients. It has been found that the active ingredients, hyoscyamine and phenyl salicylate or pharmaceutically acceptable salts thereof, are incompatible thereby adversely affecting formulations including both therein. To this end, inclusion of the barrier with respect to one or more of the incompatible ingredients reduces the “reactivity” thereby improving the stability of the formulation, and particularly in formulations comprising hyoscyamine sulfate and phenyl salicylate.

The barrier may be a physical barrier to prevent physical contact between incompatible first and second ingredients in the formulation. To this end, the barrier may be a coating comprising a material which coats or surrounds, such as an encapsulation, one of or both of the first and second ingredients prior to forming the final desired formulation. The coating or encapsulation should be inert with respect to the ingredient surrounded as well as other ingredients in the composition. Suitable coating materials include, for example, a polymeric material, such as a polyvinylpyrrolidone (PVP) polymer or other biologically acceptable materials. The coating may further comprise other desirable and beneficial materials that may improve the properties and characteristics of the coated ingredient(s) as well as the formulation as a whole. The coating also serves to reduce or eliminate “reactivity” between the coated or surrounded ingredient and other ingredients present in the formulation but not known to be incompatible with the coated or surrounded ingredient.

The present invention further provides methods of improving the stability of compositions having one or more incompatible ingredients. More particularly, the one or more incompatible ingredients are coated or surrounded with the coating material in such a manner so as to reduce reactivity therebetween or render them non-reactive in a final formulation. For example, one or more incompatible ingredients may be coated or surrounded with a coating material by dipping, spraying, brushing or by other conventional methods for applying the coating around the ingredient. Where the incompatible ingredient is a semi-solid or a liquid, it may be desirable to granulate the ingredient by first combining it with a carrier, such as lactose, to form a granulated ingredient and then coating or surrounding the granules with the coating material. To this end, the ingredient may be dissolved in a suitable medium, such as water or alcohol, and sprayed onto the carrier to granulate the ingredient. The coated or surrounded granules may be further processed or refined, such as screening for granule size, as desired to meet specific requirements for the formulation. The coated or surrounded ingredient(s) are combined with other desired ingredients including active ingredients and excipients, and formulated into a single unitary dosage form such as a liquid, a pill, tablet, a caplet, or a capsule having improved stability.

By virtue of the foregoing, there are thus provided highly stable compositions and methods for improving stability which overcome drawbacks associated with the prior art. These and other objects and advantages of the present invention shall be made apparent from the accompanying detailed description thereof.

### DETAILED DESCRIPTION OF THE INVENTION

The present invention provides pharmaceutical compositions having improved stability and methods of improving stability therein without suffering from the drawbacks and weaknesses of the prior art. To this end, and in accordance with the principles of the present invention, there is provided pharmaceutical compositions having at least two ingredients, a first ingredient of which is reactive or incompatible with a second ingredient, and a barrier therebetween, such as a coating, adapted to reduce reactivity between the first and the second ingredients in a final formulation of the composition. The barrier improves stability of the composition by reducing or eliminating reactive contact between the incompatible ingredients in the formulation. By improving the overall stability of the composition, the barrier prolongs the shelf life of the formulation.

The term “reactive”, as used herein with reference to two or more ingredients, such as a first ingredient and a second ingredient, is intended to generally refer to incompatibility between the ingredients. Accordingly, the term “reactivity” generally refers to the ability of two ingredients to interact with one another. Thus, the reactivity between ingredients may vary depending upon the degree of incompatibility. Incompatibility between ingredients is generally evident when a reaction between the ingredients results in a change, typically a detrimental change, to one or both ingredients upon physical contact. The contact generally leads to a chemical reaction wherein one or both ingredients may be chemically altered in structure and/or form, thereby having a destabilizing affect on the performance and/or function of the ingredient(s) as well as the composition as a whole. Thus, such reactive-contact between incompatible ingredients also reduces the stability of the composition and of the formulation. The degree of incompatibility or level of reactivity may be measured over time. For example, a first ingredient that is very reactive with a second ingredient may chemically destabilize the first and/or the second ingredients upon contact in a relatively short period of time, whereas, a pair of mildly reactive ingredients may require longer time periods for the same degree of degradation. The present invention is therefore applicable to incompatibilities between ingredients which may not be readily apparent but may manifest itself over time, particularly after formulating the composition. For example, a formulation comprising two incompatible ingredients may appear stable and suitable for use over a short period of time, however, over a longer duration, the mild reactivity between ingredients may be sufficient to gradually degrade one or both ingredients in the composition. Progression of such degradation may cause the final formulation to fail government stability requirements. As previously discussed herein, such failure can be costly.

The terms “first ingredient” and “second ingredient”, as used herein, are intended to refer to any two ingredients desired or necessary for inclusion in the composition. For example, the first and second ingredients may, independently, be an active ingredient or a non-active, additive ingredient, such as an excipient. The term “active ingredient”, as used herein, is intended to refer to an ingredient, or a pharmaceutically acceptable salt or derivative thereof, that produces a particular physiological change, and is, therefore, directed for the treatment of a particular symptom, ailment, disease or disorder related to the body, with minimal or no side effects. Examples of active ingredients that may be utilized in the present compositions include, without limitation, the following ingredients and/or their pharmacologically active salts: acebutolol, acetylcysteine, acetylsalicylic acid, acyclovir, alprazolam, alfacalcidol, allantoin, allopurinol, ambroxol, amikacin, amiloride, aminoacetic acid, amiodarone, amitriptyline, amlodipine, amoxicillin, ampicillin, ascorbic acid, aspartame, astemizole, atenolol, beclomethasone, benserazide, benzalkonium hydrochloride, benzocaine, benzoic acid, betamethasone, bezafibrate, biotin, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefalexin, cefadroxil, cefazolin, cefiximine, cefotaxime, ceftazidime, deftriaxone, defuroxim, selegiline, chloramphenicol, chlorhexidine, chlor-pheniramine, chlortalidone, choline, cyclosporin, cilastatin, cimetidine, ciprofloxacin, cisapride, cisplatin, clarithromycin, clavulanic acid, clomipramine, clonazepam, clonidine, clotrimazole, codeine, cholestyramine, cromoglycic acid, cyanocobalamin, cyproterone, desogestret, dexamethasone, dexpanthenol, dextromethorphan, dextropropoxiphen, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erthromycin, estradiol, ethinylestradiol, etoposide, Eucalyptus globulus, famotidine, felodipine, fenofibrate, fenoterol, fentanyl, flavine mononucleotide, fluconazole, flunarizine, fluorouracil, fluoxetine, flurbiprofen, furosemide, gallopamil, gemfibrozil, gentamicin, Gingko biloba, glibenclamide, glipizine, clozapine, Glycyrrhiza glabra, griseofulvin, fuaifenesin, haloperidol, heparin, hyaturonic acid, hydrochlorothiazide, hydrocodone, hydrocortisone, hydromorphone, hyoscyamine, ipratropium hydroxide, ibuprofen, imipenem, indomethacin, iohexol, iopamidol, isosorbide dinitrate, isosorbide mononitrate, isotretinoin, ketoifen, ketoconazole, ketoprofen, ketorolac, labetalol, lactulose, lecithin, levocarnitine, levodopa, levoglutamide, levonorgestrel, levothyroxine, lidocaine, lipase, imipramine, lisinopril, loperamide, lorazepam, lovastatin, mdroxyprogesterone, menthol, methotrexate, methyldopa, methylprednisolone, metoclopramide, metoprolol, miconazole, midazolam, minocycline, minoxidil, misoprostol, morphine, multivitamin mixtures and combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nizatidine, norethisterone, norfloxacin, norgestrel, nortriptyline, nystatin, ofloxacin, omeprazole, ondansetron, pancreatin, panthenol, pantothenic acid, paracetamol, penicillin G, penicillin V, Phenobarbital, pentoxifylline, phenoxymethylpenicillin, phenylephrine, phenylpropanolamine, phenyl salicylate, phenytoin, piroxicam, polymyxin B, povidone iodine, pravastatin, prazepam, prazosin, prednisolone, prednisone, bromocriptine, propafenone, propranolol, proxyphylline, pseudoephedrine, pyridoxine, quinidine, ramipril, ranitidine, reserpine, retinal, riboflavin, rifampicin, rutoside, saccharin, salbutamol, salcatonin, salicylic acid, simvastatin, comatropin, sotalol, spironolactone, sucralfate, sulbactam, sulfamethoxazole, sulfasalazine, sulpiride, tamoxifen, tegafur, teprenone, terazosin, terbutaline, terpenadine, tetracycline, theophylline, thiamine, ticlopidine, timolol, tranexamic acid, tretinoin, triamcinolone acetonide, triamterene, trimethoprim, troxerutin, uracil, valproic acid, vancomycin, verapamil, folinic acid, and zidovudine.

The active ingredient may also be an analgesic, such as ibuprofen (as racemate, enantiomer or enriched enantiomer), ketoprofen, flurbiprofen, acetylsalicylic acid, cerapamil, paracetamol, nifedipine, captopril, omeprazole, ranitidine, tramadol, cyclosporin, trandolapril or a therapeutic peptide.

The first or second ingredients, independently, is not limited to active ingredients, and may be a vitamin. Examples of vitamins include, without limitation, vitamins of the A group, the B group, such as for example vitamins B₁, B₂, B₆, B₁₂, nicotinic acid, and nicotinimide as well as compounds with vitamin B properties such as adonine, choline, pantothenic acid, biotin, adenylic acid, folic acid, orotic acid, pangamic acid, carnitine, p-aminobenzoic acid, myo-inositol and lipoic acid, vitamin C, vitamins of the D group, E group, F group, H group, I and J group, K group and P group.

While the above lists of pharmaceutical ingredients are quite extensive, the first and second ingredients contemplated by the present invention are not so limited and the first and second ingredients may, independently, be other ingredients not included in the lists above. For example, the first and second ingredients may, independently, be an excipient. Conventionally accepted expedients include for example, pharmaceutical accelerators, extenders, and bulking agents such as silicates or diatomaceous earth, magnesium oxide, aluminum, titanium oxide, methyl cellulose, sodium, carboxymethyl cellulose talc, sucrose, lactose, cereal or cornstarch, potato flour, and polyvinylalcohol. Lubricants as a release agent such as magnesium, aluminum and calcium stearate talc, silicones and animal/vegetable fats, especially in hydrogenated form, and those which are solid at room temperature may also be included. Suitable auxiliaries include flow regulators, dyes, such as artificial dyes or dyes or natural origin, organic or inorganic pigments, stabilizers such as antioxidants, light stabilizers, hydroperoxide destroyers, radical scavengers, stabilizers against microbial attack, wetting agents, preservatives, disintegrants, absorbants, mold release agents and propellants. Auxiliaries also means for the purpose of the invention, substances for producing a solid or a solution containing the pharmaceutical active ingredient. Pharmaceutical auxiliaries are also regarded as being bases and acids added to control solubility of an active ingredient. Regardless of type or class of the ingredients, a first ingredient that is reactive with a second ingredient, and otherwise incompatible the second ingredient, may be formulated with the barrier of the present invention for improving stability of the formulation. Thus, the incompatibility addressed by the present invention may manifest itself between an expedient and one or more of the active ingredients.

The incompatible first and second ingredients are rendered less reactive or even non-reactive within the composition, and the final formulation, by inclusion of a barrier with respect to one or both of the first and second ingredients. The barrier is a physical barrier around one or both of the incompatible ingredients thereby reducing reactivity therebetween in the composition. In one embodiment of the present invention, the barrier is a coating around at least one of the first and second ingredients. The coating serves to reduce or eliminates physical contact between the coated ingredient and other ingredients, which may be incompatible therewith, in the composition. To this end, the coating may completely enclose or surround the ingredient. The ingredient may be coated in a variety of ways. In one embodiment, the ingredient is coated directly with a coating material adhered to the surface of the ingredient. In another embodiment, the coating material encapsulates the incompatible ingredient to reduce its reactivity with other ingredients in the composition.

The coating comprises a coating material, which should be biologically compatible. In one embodiment of the invention, the coating material is a polymeric material. Many polymer materials are known to be biologically acceptable and inert with respect to desirable ingredients, and particularly active ingredients. Suitable polymeric coatings include, without limitation, single polymers of varying length and molecular weight. For example, the coating material may comprise a poly(N-viny lactam) polymer, such as a polyvinylpyrrolidone polymer (PVP). A suitable form of PVP is commercially available as ‘Povidone K-90’ from BASF Corporation, New Jersey, USA. The coating material may also be a copolymer of different monomeric units. Suitable copolymer blends include a first polymeric component, such as an inorganic solvent-soluble thermoplastic polyurethane, and a second polymer component, such as water soluble, hydrophilic poly(N-viny lactam), to provide a low friction coating. Such polymers and copolymers are described in further detail in U.S. Pat. No. 4,642,267, the disclosure of which is incorporated herein by reference in its entirety. Other suitable polymeric coating materials include, but are not limited to, biodegradable and biocompatible polymers, polysaccharides, and proteins. Suitable biodegradable polymers include polylactides, polyglycolides, polycaprolactones, polydioxonones, polycarbonates, polyhydroxybutylates, polyalkylene oxalates, polyanhydrides, polyamides, polyester amides, polyurethanes, polyacetates, polyketals, polyorthocarbonates, polyphosphosines, polyhydroxy valerates, polyalkylene succinates, poly(maleic acid), poly(amino acids), polyvinylpyrrolidone, polyethylene glycol, polyhydroxy cellulose, polyorthoesters, and combinations thereof, as well as polylactic acids, polymers and copolymers, polyorthoesters, and polycaprolactones, etc. Suitable biocompatible polymers include, for example, polyethyleneglycols, polyvinylpyrrolidones, and polyvinylalcohols, etc. Suitable polysachharides include dextrans, cellulose, xantham, chitin, and chitosans, etc. Suitable proteins include polylysines and other polyamines, collagen, albumen, etc. A number of materials particularly useful as coating materials are enclosed in U.S. Pat. No. 5,702,716, the disclosure of which is incorporated herein by reference in its entirety. Further, alkyl cellulostic polymers are suitable for surrounding water reactive ingredients, as disclosed in U.S. Pat. No. 6,258,323, the disclosure of which is incorporated herein by reference in its entirety. The coating material should comprise only those materials that are not reactive with either or both of the coated or surrounded ingredient(s) and other ingredients included in the composition.

The barrier of the present invention, such as a coating, is useful for improving the stability of compositions and formulations including hyoscyamine or a pharmaceutically acceptable salt thereof, such as hyoscyamine sulfate, and phenyl salicylate or a pharmaceutically acceptable salt thereof, known to be otherwise incompatible with one another in a single composition or formulation. In one embodiment of the present invention, a PVP coating surrounds at least one of hyoscyamine sulfate and phenyl salicylate in a formulation to enhance stability and prolong the shelf life of the formulation. In yet another embodiment, a PVP coating surrounds both the hyoscyamine sulfate and phenyl salicylate in the formulation to improve stability therein.

An ingredient may be coated with a coating material by a number of conventional techniques. As described above, the ingredient may be coated directly or indirectly, by encapsulation, with the coating material. Suitable direct coating methods include, without limitation, spraying, dipping, brushing or other methods that may partially or completely coat the ingredient with the coating material. Coating via conventional encapsulation techniques are also contemplated. For example, solvent evaporation techniques used for encapsulating an ingredient with a coating material may also be used. Microencapsulation, a technique more recently developed, may also be used.

Solid or particulate forms of the ingredient are easily and conveniently coated with the coating material by simply spraying the ingredient in a fluid bed with a liquid form or solution of the coating material. Spray-drying techniques are cost effective for solid forms of the ingredient. Where the ingredient is a semi-solid or a liquid, it may advantageously be granulated into a solid particulate form with the aid of a carrier. Further, solid forms of an ingredient may be converted to a liquid and sprayed onto a carrier for improved distribution and/or even dispersion of the ingredient within the formulation. The term “carrier”, as used herein, is intended to refer to an inert, solid vehicle for solidifying an otherwise non-solid form of an ingredient. The carrier may be any known vehicle that is biologically acceptable, chemically inert, and suitable for granulating the desired ingredient for coating thereon. For example, the carrier may be a carbohydrate, such as a monosaccharide, a disaccharide, or a polysaccharide, or a polyhydroxy compound. Suitable carbohydrate carriers include, without limitation, one or a combination of dextrose, glucose, sucrose, arabinose, lactose, mannose, maltose, fructose, galactose, amylose, allose, altose, talose, gulose, idose, ribose, erythrose, threose, lyxose, xylose, and rhamnose. Suitable polyhydroxy compounds include, without limitation, inositol, xylitol, mannitol, sorbitol, lactitol, maltitol, isomalt, and glycerol. In one embodiment of the present invention, the carrier is lactose. The semi-solid or non-solid ingredient may be blended with or dissolved into a suitable or desired solvent, such as water or alcohol, to form a solution or suspension that may be applied, such as by spraying, onto the carrier. In this manner, the semi-solid or non-solid ingredient may be uniformly distributed on the carrier and, therefore, evenly distributed in the final formulation. The granulated ingredient may then be coated by methods described herein. In one embodiment of the present invention, a liquid form of hyoscyamine, such as a solution of hyoscyamine sulfate, is sprayed onto lactose to produce a granular form of the hyoscamine, whereafter the individual granules of hyoscyamine are coated with a polyvinylpyrrolidone (PVP) polymer coating material, Povidone K-90. The PVP coating provides a physical barrier, impenetrable by other liquids and/or solids incompatible with hyoscyamine and present in the composition thereby reducing or eliminating reactivity with hyoscyamine. Such an improvement in stability of the hyoscamine is likely to allow a composition or formulation having hyoscamine contained therein to meet and possibly exceed federal stability standards. In another embodiment, a formulation including the incompatible ingredients, hyoscyamine and phenyl salicylate, is prepared in the following manner. First, hyoscamine sulfate and a PVP polymer solution are combined and dissolved in water. The aqueous PVP-hyoscamine solution is sprayed onto a lactose carrier to granulate the hyoscamine for inclusion in the formulation. The hyoscamine granules are loaded into a fluid bed and sprayed with a PVP polymer solution to coat the granules. The coated granular hyoscamine is combined with similarly coated phenyl salicylate into a pharmaceutically acceptable formulation.

The barrier around the ingredient, such as the coating described above, also serves to reduce reactivity or render the ingredient non-reactive with elements of the environment. As described earlier, humidity can play a role in reducing stability of both an ingredient and a formulation. Thus, ingredients reactive to water may be surrounded with a hydrophobic coating, for example, for preventing contact with water or moisture in the air. The water resistant coating material may also provide a water resistant barrier around the ingredient preventing moisture or water induced decomposition or reactions with other ingredients in the formulation.

The coated ingredient may then be further processed as desired prior to final formulation of the composition. Conventional processing techniques may be utilized. For example, where the ingredient size or amount is critical to the formulation, the coated ingredient, or granular particles, may be screened for size so as to limit the size and better distribute the ingredient more evenly in the composition. Screening, therefore, provides blend or content uniformity of ingredients in the formulation. Screening is also useful as a “pre-screen” to limit granular particle size as desired even prior to coating the ingredient with a coating material. Screening may be accomplished by using a screen, for example, of desired mesh, such as about 20-40 mesh, to filter out particles of undesirable size and prevent them from being included into the final dosage form. Thus, particles having a diameter of about 850 μM or less may be collected through a screen of 20 mesh. Similarly, particles having a diameter of about 600 μM or less may be collected through a screen of 30 mesh, and of about 425 μM or less may be collected through a screen of 40 mesh. In one embodiment, granular hyoscamine, after coating with a coating material and drying, is filtered through a screen having a size of about 30 mesh, to obtain granules having a diameter of about 600 μM or less, prior to combination with phenyl salicylate in a final formulation. In another embodiment, both hyoscamine and phenyl salicylate are independently filtered through a screen of about 30 mesh prior to final formulation.

In another aspect of the present invention, the coating material may coat each and every known incompatible ingredient in the formulation. Coating all incompatible ingredients practically guarantees protection against degradation resulting from the reactivity and incompatibility therebetween. Particularly, where two incompatible ingredients are active ingredients, coating both ingredients provides the advantage of minimizing or eliminating reactions between an active ingredient and another ingredient deemed non-reactive or otherwise not known to be reactive with that active ingredient in the formulation. Thus, coating all incompatible ingredients provides an advantage or a safely net against unknown incompatibilities between them and other ingredients in the formulation. Accordingly, without significant additional costs, the present invention prolongs the shelf life of compositions formulated with incompatible ingredients.

The pharmaceutical composition of the present invention is formulated into a suitable form. For example, the composition may be formulated into an ingestible form, such as a liquid, a pill, a tablet, a caplet, or a capsule. By way of example, the tablet may be a soft, chewable tablet amenable to children. The capsule may be a hard or soft shell capsule, including gelatin capsules. Soft-gel capsules are popular and convenient. Further, the present formulation contemplates film encapsulation of a liquid form of the composition. Conventional techniques and methods may be used to produce these forms. It will be appreciated by persons of ordinary skill in the art of formulation that a particular or desired formulation may be limited depending on the type, amount, and concentration of ingredients, including those coated with a coating material, present in the formulation. In addition to the first and second ingredients, or any other incompatible ingredients, it is desirable to include many useful excipients for rendering the formulation with desirable properties and aesthetics. By way of example, suitable excipients include a lubricating agent, a disintegrating agent, a delivery agent, a dissolution agent, a colorant, a fragrance, a texture modifying agent, a flavoring agent, a flow agent, and combinations thereof. Persons of ordinary skill in the art of formulations appreciate inclusion of such agents. For example, a delivery agent, such as a coating around the entire formulation itself, may be used where the formulation is designed to control activity and rate of release of an ingredient, including the incompatible ingredients. A biologically acceptable coating may help to delay release of an ingredient or to improve absorption of one or more ingredients into the blood. In addition to useful ingestible formulations, the present invention is also applicable to formulations not intended for ingestion or consumption by a mammal, but having incompatible ingredients therein. For example, the present invention is also applicable to dermal patches containing incompatible reagents.

Thus, the present invention provides compositions containing incompatible ingredients, and particularly active ingredients, with improved stability and methods of improving stability thereto in a simple and cost effective manner, while addressing the drawbacks of the prior art. The present invention does so by providing a barrier between the ingredients, such as a biologically acceptable coating around the individual ingredient(s), thereby reducing the ability of incompatible ingredients to react with one another, or generally rendering them non-reactive in the formulation. The coating may also be an encapsulation around the ingredient, provided prior to formulation of the composition. All incompatible ingredients in the composition may be coated to practically ensure an improvement in stability thereof. The improvement in stability, and therefore the shelf life, should be sufficient, in many cases, to satisfy government stability requirements, and in particular to meet the USP and the FDA stability standards.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative apparatus and method, and illustrated examples shown and described. Accordingly, departures may be made from such details without departing from the scope or spirit of Applicants' general inventive concept.

What is claimed is:

1. A pharmaceutical composition comprising at least two ingredients of which a first ingredient is reactive with a second ingredient, the composition in a formulation wherein at least one of the first ingredient and the second ingredient further contains a coating thereby reducing the reactivity between the ingredients.

2. The composition of claim 1 wherein the coating comprises a polymeric material.

3. The composition of claim 1 wherein the coating comprises a polyvinyl pyrrolidone polymer.

4. The composition of claim 1 wherein the first ingredient contains a coating and the second ingredient contains a coating.

5. The composition of claim 1 wherein the coating is an encapsulation of at least one of the first ingredient and the second ingredient.

6. The composition of claim 1 wherein the coating prevents physical contact between the first ingredient and the second ingredient in the formulation.

7. The composition of claim 1 wherein at least one of the first ingredient and the second ingredient is a pharmaceutically active ingredient.

8. The composition of claim 1 wherein the first ingredient is hyoscyamine or a pharmaceutically acceptable salt thereof.

9. The composition of claim 1 wherein the second ingredient is phenyl salicylate.

10. The composition of claim 1 wherein at least one of the first ingredient and the second ingredient is a granular material wherein each granule comprises a carrier.

11. The composition of claim 10 wherein the carrier is selected from the group consisting of dextrose, glucose, sucrose, arabinose, lactose, mannose, maltose, fructose, galactose, amylose, allose, altose, talose, gulose, idose, ribose, erythrose, threose, lyxose, xylose, rhamnose, inositol, xylitol, mannitol, sorbitol, lactitol, maltitol, isomalt, glycerol, and combinations thereof.

12. The composition of claim 1 wherein at least one of the first ingredient and the second ingredient is evenly dispersed within the formulation.

13. The composition of claim 1 wherein the formulation is selected from the group consisting of a liquid, a pill, a tablet, a caplet, and a capsule.

14. The formulation of claim 1 further comprising at least one excipient selected from the group consisting of a lubricating agent, a disintegrating agent, a delivery agent, a dissolution agent, a colorant, a fragrance, a texture modifying agent, a flavoring agent, a flow agent, and combinations thereof.

15. A pharmaceutical composition comprising hyoscyamine and phenyl salicylate in a formulation wherein at least one of the hyoscyamine and the phenyl salicylate contain a barrier to physical contact with the other thereby reducing the reactivity between the hyoscyamine and the phenyl salicylate in the formulation.

16. The composition of claim 15 wherein the barrier is a coating.

17. The composition of claim 16 wherein the hyoscyamine contains a coating and the phenyl salicylate contains a coating.

18. The composition of claim 16 wherein the coating comprises a polymeric material.

19. The composition of claim 16 wherein the coating comprises a polyvinyl pyrrolidone polymer.

20. The composition of claim 16 wherein the coating is an encapsulation of at least one of the hyoscyamine and the phenyl salicylate.

21. The composition of claim 16 wherein the coating prevents physical contact between the hyoscyamine and the phenyl salicylate in the formulation.

22. The composition of claim 15 wherein the hyoscyamine is a granular material wherein each granule comprises a carrier.

23. The composition of claim 22 wherein the carrier is selected from the group consisting of dextrose, glucose, sucrose, arabinose, lactose, mannose, maltose, fructose, galactose, amylose, allose, altose, talose, gulose, idose, ribose, erythrose, threose, lyxose, xylose, rhamnose, inositol, xylitol, mannitol, sorbitol, lactitol, maltitol, isomalt, glycerol, and combinations thereof.

24. The composition of claim 15 wherein at least one of the hyoscyamine and the phenyl salicylate is evenly dispersed within the formulation.

25. The composition of claim 15 wherein the formulation is selected from the group consisting of a liquid, a pill, a tablet, a caplet, and a capsule.

26. The composition of claim 15 further comprising at least one excipient selected from the group consisting of a lubricating agent, a disintegrating agent, a delivery agent, a dissolution agent, a colorant, a fragrance, a texture modifying agent, a flavoring agent, a flow agent, and combinations thereof.

27. The composition of claim 15 further comprising methylene blue, methenamine, and sodium phosphate.

28. A pharmaceutical composition comprising granules of hyoscyamine evenly dispersed with phenyl salicylate in a solid formulation wherein the hyoscyamine contains a polymeric coating thereby reducing reactivity between the hyoscyamine and the phenyl salicylate.

29. The composition of claim 28 wherein the formulation is a tablet.

30. The composition of claim 28 wherein the coating comprises a polyvinyl pyrrolidone polymer.

31. The composition of claim 28 wherein each hyoscyamine granule comprises a carrier selected from the group consisting of dextrose, glucose, sucrose, arabinose, lactose, mannose, maltose, fructose, galactose, amylose, allose, altose, talose, gulose, idose, ribose, erythrose, threose, lyxose, xylose, rhamnose, inositol, xylitol, mannitol, sorbitol, lactitol, maltitol, isomalt, glycerol, and combinations thereof.

32. The composition of claim 31 wherein each hyoscyamine granule has a size of about 600 μM or less.

33. The composition of claim 28 wherein the phenyl salicylate is a particulate solid having a particle size of about 600 μM or less.

34. A method for improving the stability of a pharmaceutical composition, the method comprising:
providing at least two ingredients for inclusion in the composition, a first ingredient of which is reactive with a second ingredient;
coating at least one of the first and second ingredients with a coating material independently of the other ingredient; and
formulating the ingredients whereby the coating reduces reactivity between the ingredients thereby improving the stability of the composition.

35. The method of claim 34 wherein coating at least one of the first ingredient and the second ingredient with a coating material comprises encapsulating the at least one of the first ingredient and the second ingredient with the coating material.

36. The method of claim 34 wherein the coating material comprises a polymeric material.

37. The method of claim 34 wherein the coating material comprises a polyvinylpyrrolidone polymer.

38. The method of claim 34 wherein at least one of the first ingredient and the second ingredient is a pharmaceutically active ingredient.

39. The method of claim 34 wherein the first ingredient is hyoscyamine or a pharmaceutically acceptable salt thereof.

40. The method of claim 34 wherein the second ingredient is phenyl salicylate.

41. The method of claim 34 further comprising evenly dispersing at least one of the first ingredient and the second ingredient during formulation.

42. The method of claim 34 wherein coating the at least one of the first ingredient and the second ingredient comprises spraying the at least one of the first ingredient and the second ingredient with a liquid form of the coating material.

43. The method of claim 34 further comprising granulating at least one of the first ingredient and the second ingredient with a carrier prior to coating the at least one of the first ingredient and the second ingredient with a coating material.

44. The method of claim 43 wherein the carrier is selected from the group consisting of dextrose, glucose, sucrose, arabinose, lactose, mannose, maltose, fructose, galactose, amylose, allose, altose, talose, gulose, idose, ribose, erythrose, threose, lyxose, xylose, rhamnose, inositol, xylitol, mannitol, sorbitol, lactitol, maltitol, isomalt, glycerol, and combinations thereof.

45. The method of claim 43 wherein granulating the at least one of the first ingredient and the second ingredient with a carrier comprises spraying a liquid form of the at least one of the first ingredient and the second ingredient onto the carrier.

46. The method of claim 43 further comprising screening the granulated at least one of the first ingredient and the second ingredient through a mesh prior to coating with a coating material.

47. The method of claim 34 further comprising screening the coated at least one of the first ingredient and the second ingredient through a mesh prior to formulating the ingredients to form the stable composition.

48. The method of claim 34 wherein the ingredients are formulated into a form selected from the group consisting of a liquid, a pill, a tablet, a caplet, and a capsule.

49. A method for improving the stability of a pharmaceutical composition, the method comprising:
providing hyoscyamine or a pharmaceutically acceptable salt thereof and phenyl salicylate for inclusion in the composition;
coating at least one of the hyoscyamine and the phenyl salicylate with a coating material; and
dispersing the hyoscyamine evenly with the phenyl salicylate in the composition wherein the coating material reduces reactivity between the hyoscyamine and the phenyl salicylate for improving stability of the composition.

50. The method of claim 49 wherein coating at least one of the hyoscyamine and the phenyl salicylate with a coating material comprises encapsulating the at least one of the hyoscyamine and the phenyl salicylate with the coating material.

51. The method of claim 49 wherein the coating material comprises a polymeric material.

52. The method of claim 49 wherein the coating material comprises a polyvinylpyrrolidone polymer.

53. The method of claim 49 wherein coating the at least one of the hyoscyamine and the phenyl salicylate comprises spraying the at least one of the hyoscyamine and the phenyl salicylate with a liquid form of the coating material for coating thereon.

54. The method of claim 49 further comprising granulating at least one of the hyoscyamine and the phenyl salicylate with a carrier prior to coating the at least one of the hyoscyamine and the phenyl salicylate with a coating material.

55. The method of claim 54 wherein the carrier is selected from the group consisting of dextrose, glucose, sucrose, arabinose, lactose, mannose, maltose, fructose, galactose, amylose, allose, altose, talose, gulose, idose, ribose, erythrose, threose, lyxose, xylose, rhamnose, inositol, xylitol, mannitol, sorbitol, lactitol, maltitol, isomalt, glycerol, and combinations thereof.

56. The method of claim 54 wherein granulating the at least one of the hyoscyamine and the phenyl salicylate with a carrier comprises spraying the at least one of hyoscyamine and the phenyl salicylate onto the carrier.

57. The method of claim 54 further comprising screening the granulated at least one of the hyoscyamine and the phenyl salicylate through a mesh prior to coating with a coating material.

58. The method of claim 49 wherein the ingredients are formulated into a form selected from the group consisting of a liquid, a pill, a tablet, a caplet, and a capsule.

59. A formulation method for preventing a reaction between a first active ingredient and a second active ingredient in a formulation, the method comprising
combining a solution of the first active ingredient with a carrier to form granular first active ingredient;
coating the granular first active ingredient with a coating material;
drying the coated first active ingredient; and
formulating the coated first active ingredient with the second active ingredient whereby the first active ingredient is rendered non-reactive with the second ingredient in the formulation.

60. The method of claim 59 further comprising coating the second active ingredient with a coating material prior to formulating with the first active ingredient.

61. A pharmaceutical formulation formed by the process of:
providing hyoscyamine or a pharmaceutically acceptable salt thereof and phenyl salicylate for inclusion in the formulation;
coating at least one of the hyoscyamine and the phenyl salicylate with a coating material; and
evenly dispersing the hyoscyamine and phenyl salicylate in the formulation whereby the coating material reduces reactivity between the hyoscyamine and the phenyl salicylate.
